(19) United States
(12) Patent Application Publication
(10) Pub. No.: US 2017/0266293 A1
(43) Pub. Date: Sep. 21, 2017

(54) METHODS OF TREATING CANCERS WITH THERAPEUTIC NANOPARTICLES

(71) Applicant: PFIZER INC., New York, NY (US)

(72) Inventors: STEPHEN E. ZALE, HOPKINTON, MA (US); GREG TROIANO, PEMBROKE, MA (US); MIR MUKKARAM ALI, WOBURN, MA (US); JEFF HRKACH, LEXINGTON, MA (US); JAMES WRIGHT, LEXINGTON, MA (US)

(73) Assignee: PFIZER INC., New York, NY (US)

(21) Appl. No.: 15/603,151

(22) Filed: May 23, 2017

Related U.S. Application Data
(62) Division of application No. 14/346,456, filed on Mar. 21, 2014.
(60) Provisional application No. 61/537,980, filed on Sep. 22, 2011.

(51) Int. Cl. A61K 47/34; A61K 9/00; A61K 9/51; A61K 31/337

(52) U.S. Cl. CPC A61K 47/34; A61K 31/337; A61K 9/5153; A61K 9/0019

(57) ABSTRACT

The present disclosure relates in part to methods of treating cholangiocarcinoma or tonsillar cancer in a patient in need thereof, comprising administering to the patient a therapeutically effective amount of a nanoparticle composition, wherein nanoparticle composition comprises nanoparticles.

### CROSS REFERENCE TO RELATED APPLICATIONS

This application is a divisional application of U.S. application Ser. No. 14/346,456, filed Mar. 21, 2014, which is the National Stage Entry of Internal (PCT) Patent Application Serial No. PCT/US2012/056891, filed Sep. 24, 2012, which in turn claims priority to United States Provisional Patent Application. 61/537,980, filed Sep. 22, 2011, each of the foregoing applications is hereby incorporated by reference in its entirety.

### BACKGROUND

Systems that deliver certain drugs to a patient (e.g., targeted to a particular tissue or cell type or targeted to a specific diseased tissue but not normal tissue), or that control release of drugs has long been recognized as beneficial.

For example, therapeutics that include an active drug and that are e.g., targeted to a particular tissue or cell type or targeted to a specific diseased tissue but not to normal tissue, may reduce the amount of the drug in tissues of the body that are not targeted. This is particularly important when treating a condition such as cancer where it is desirable that a cytotoxic dose of the drug is delivered to cancer cells without killing the surrounding non-cancerous tissue. Effective drug targeting may reduce the undesirable and sometimes life threatening side effects common in anticancer therapy. In addition, such therapeutics may allow drugs to reach certain tissues they would otherwise be unable to reach.

Therapeutics that offer controlled release and/or targeted therapy also must be able to deliver an effective amount of drug, which is a known limitation in other nanoparticle delivery systems. For example, it can be a challenge to prepare nanoparticle systems that have an appropriate amount of drug associated each nanoparticle, while keeping the size of the nanoparticles small enough to have advantageous delivery properties. However, while it is desirable to load a nanoparticle with a high quantity of therapeutic agent, nanoparticle preparations that use a drug load that is too high will result in nanoparticles that are too large for practical therapeutic use.

Accordingly, a need exists for nanoparticle therapeutics and methods of making such nanoparticles, that are capable of delivering therapeutic levels of drug to treat diseases such as cancer, while also reducing patient side effects.

### SUMMARY

In one aspect, the disclosure provides a method of treating certain cancers such as cancers of lymph or biliary ducts, (e.g. cholangiocarcinoma, pancreatic cancer, gallbladder cancer, and/or cancer of the ampulla of Vater), comprising administering to a patient in need thereof a composition comprising a disclosed therapeutic nanoparticle (e.g. a nanoparticle comprising a therapeutic agent (e.g. docetaxel) and a biocompatible polymer). In another aspect, the invention provides a method of treating certain cancers such as oropharnx cancers or cancers of the throat, e.g. tonsillar cancer, comprising administering to a patient in need thereof: a composition comprising a disclosed therapeutic nanoparticle (e.g. a nanoparticle comprising a therapeutic agent (e.g. docetaxel) and a biocompatible polymer). For example, disclosed nanoparticle may include an active agent or therapeutic agent, e.g. taxane (e.g. docetaxel) and a biocompatible polymer. For example, disclosed herein is a therapeutic nanoparticle comprising about 0.2 to about 35 weight percent of a therapeutic agent; about 10 to about 99 weight percent poly(lactic) acid-block-poly(ethylene)glycol copolymer or poly(lactic)-co-poly (glycolic) acid-block-poly(ethylene)glycol copolymer. The hydrodynamic diameter of disclosed nanoparticles may be, for example, about 60 to about 150 nm, or about 70 to about 120 nm. Such poly(lactic) acid-block-poly(ethylene)glycol copolymer may include poly(lactic acid) having a number average molecular weight of about 15 to 20 kDa and poly(ethylene)glycol having a number average molecular weight of about 4 to about 6 kDa. In some embodiments, disclosed nanoparticles may further comprise about 0.2 to about 10 weight percent PLA-PEG functionalized with a targeting ligand and/or may include about 0.2 to about 10 weight percent poly (lactic) acid-co poly (glycolic) acid block-PEG-functionalized with a targeting ligand. Such a targeting ligand may be, in some embodiments, covalently bound to the PEG, for example, bound to the PEG via an alkylene linker, e.g. PLA-PEG-alkylene-GL2, wherein the alkylene is e.g. C₁-C₂₀, e.g., (CH₂)₅, linking the PEG to GL2.

For example, provided herein is a method of treating cholangiocarcinoma or tonsillar cancer in a patient in need thereof, comprising administering to the patient a therapeutically effective amount of a nanoparticle composition (e.g. a pharmaceutically acceptable composition comprising a disclosed nanoparticle), wherein nanoparticle composition comprises nanoparticles having a hydrodynamic diameter of about 60 to about 150 nm and the nanoparticles comprise docetaxel and about 10 to about 97 weight percent of a diblock poly(lactic) acid-poly(ethylene)glycol copolymer, wherein the poly(lactic acid) block has a number average molecular weight of about 15 to 20 kDa and the poly(ethylene)glycol block has a number average molecular weight of about 4 to about 6 kDa.

In some embodiments, a disclosed method includes administering a therapeutically effective amount of a disclosed nanoparticle composition, wherein the nanoparticle composition has about 50 to about 75 mg/m² of docetaxel, or about 60 to about 75 mg/m² of docetaxel, or e.g. about 60 mg/m² of docetaxel.

A disclosed method, for example, may include comprising administering a disclosed composition about every three weeks to said patient, e.g. administered by intravenous infusion over about 1 hour.

In some embodiments, a disclosed method is directed to treating a cancer such as cholangiocarcinoma or tonsillar cancer, where the cancer was not stabilized with another chemotherapeutic agent or combination of chemotherapeutic agents after previous administration to the patient.

Also provided herein is a method of treating a refractory cancer in a patient in need thereof, comprising administering to the patient a therapeutically effective amount of nanoparticle composition, wherein nanoparticle composition comprises nanoparticles having a hydrodynamic diameter of about 60 to about 130 nm comprising: a chemotherapeutic agent and about 10 to about 97 weight percent of a diblock poly(lactic) acid-poly(ethylene)glycol copolymer, wherein the poly(lactic acid) block has a number average molecular weight of about 15 to 20 kDa and the poly(ethylene)glycol block has a number average molecular weight of about 4 to about 6 kDa, wherein the refractory cancer is refractory to other chemotherapy and/or radiation therapy alone. Contemplated refractory cancers include gastrointestinal cancer or oropharyngeal cancer, or e.g., tonsillar cancer, anal cancer, pancreatic cancer, bile duct cancer. Refractory cancers contemplated herein include cervical cancer, lung cancer, and prostate cancer.

### BRIEF DESCRIPTION OF THE DRAWINGS

FIG. 1 shows a baseline scan (left panel) and a post treatment scan (right panel) of a human cholangiocarcinoma patient before and about 40 days after treatment with a composition including a disclosed nanoparticle composition.

FIG. 2 shows a baseline scan (left panel) and a post treatment scan (right panel) of a human cholangiocarcinoma patient before and about 40 days after treatment with a composition including a disclosed nanoparticle composition.

FIG. 3 shows a baseline scan (left panel) and a post treatment scan (right panel) of a human tonsillar cancer patient before and about 40 days after treatment with a composition including a disclosed nanoparticle composition.

FIG. 4 shows results of a pharmacokinetics (PK) study using a disclosed nanoparticle composition.

FIG. 5 shows plots demonstrating PK linearity.

FIG. 6 indicates the that sustained docetaxel exposure after administration of disclosed nanoparticle compositions providing about 75 mg/m² docetaxel to a patient has sustained exposure as compared to administering the same amount of docetaxel alone.

### DETAILED DESCRIPTION

The present disclosure generally relates to methods of treating various cancers by administering polymeric nanoparticles that include an active or therapeutic agent or drug. In general, a “nanoparticle” refers to any particle having a diameter of less than 1000 nm, e.g. about 10 nm to about 200 nm. Disclosed therapeutic nanoparticles may include nanoparticles having a diameter of about 60 to about 120 nm, about 70 to about 120 nm or about 70 to about 130 nm, or about 60 to about 140 nm.

Disclosed nanoparticles may include about 0.2 to about 35 weight percent, about 3 to about 40 weight percent, about 5 to about 30 weight percent, 10 to about 30 weight percent, 15 to 25 weight percent, or even about 4 to about 25 weight percent of an active agent, such as antineoplastic agent, e.g. a taxane agent (for example docetaxel). In an embodiment, an active or therapeutic agent may (or may not be) conjugated to e.g. a disclosed polymer that forms part of a disclosed nanoparticle, e.g., an active agent may be conjugated (e.g. covalently bound, e.g. directly or through a linking moiety) to PLA or PGLA, or a PLA or PLGA portion of a copolymer such as PLA-PEG or PLGA-PEG. In other embodiments, a disclosed nanoparticle may include two or more active agents.

In one embodiment, disclosed therapeutic nanoparticles may include a targeting ligand, e.g., a low-molecular weight PSMA ligand effective for the treatment of a disease or disorder, such as prostate cancer, in a subject in need thereof. In certain embodiments, the low-molecular weight ligand is conjugated to a polymer, and the nanoparticle comprises a certain ratio of ligand-conjugated polymer(e.g., PLA-PEG-Ligand) to non-functionalized polymer (e.g. PLA-PEG or PLGA-PEG). The nanoparticle can have an optimized ratio of these two polymers such that an effective amount of ligand is associated with the nanoparticle for treatment of a disease or disorder, such as cancer. For example, an increased ligand density may increase target binding (cell binding/target uptake), making the nanoparticle “target specific.” Alternatively, a certain concentration of non-functionalized polymer (e.g., non-functionalized PLGA-PEG copolymer) in the nanoparticle can control inflammation and/or immunogenicity (i.e., the ability to provoke an immune response), and allow the nanoparticle to have a circulation half-life that is adequate for the treatment of a disease or disorder (e.g., to prostate cancer). Furthermore, the non-functionalized polymer may, in some embodiments, lower the rate of clearance from the circulatory system via the reticuloendothelial system (RES). Thus, the non-functionalized polymer may provide the nanoparticle with characteristics that may allow the particle to travel through the body upon administration. In some embodiments, a non-functionalized polymer may balance an otherwise high concentration of ligands, which can otherwise accelerate clearance by the subject, resulting in less delivery to the target cells.

### Nanoparticles

Disclosed nanoparticles comprise a matrix of polymers and at least one therapeutic agent. In some embodiments, a therapeutic agent and/or targeting moiety (i.e., a low-molecular weight PSMA ligand) can be associated with at least part of the polymeric matrix. For example, in some embodiments, a targeting moiety (e.g. ligand) can be covalently associated with the surface of a polymeric matrix. In some embodiments, covalent association is mediated by a linker. The therapeutic agent can be associated with the surface of, encapsulated within, surrounded by, and/or dispersed throughout the polymeric matrix.

The term “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. Non-limiting examples include peptides or proteins. In some cases, additional moieties may also be present in the polymer, for example biological moieties such as those described below. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

Disclosed particles can include copolymers, which, in some embodiments, describes two or more polymers (such as those described herein) that have been associated with each other, usually by covalent bonding of the two or more polymers together. Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In one set of embodiments, a polymer (e.g., copolymer, e.g., block copolymer) contemplated herein includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the therapeutic particles contemplated herein can be non-immunogenic. The term non-immunogenic as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such.

In certain embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

Contemplated nanoparticles polyesters, for example, copolymers and/or block copolymers comprising lactic acid and/or glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEGylated polymers and copolymers of lactide and glycolide (e.g., PEGylated PLA (PLA-PEG), PEGylated PGA, PEGylated PLGA, and derivatives thereof.

In some embodiments, a contemplated nanoparticle may include PLGA. PLGA is a biocompatible and biodegradable co-polymer of lactic acid and glycolic acid, and various forms of PLGA can be characterized by the ratio of lactic acid:glycolic acid. Lactic acid can be L-lactic acid, D-lactic acid, or D,L-lactic acid. The degradation rate of PLGA can be adjusted by altering the lactic acid-glycolic acid ratio. In some embodiments, PLGA to be used in accordance with the present invention can be characterized by a lactic acid:glycolic acid ratio of approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., the PLGA block copolymer or PLGA-PEG block copolymer), may be selected to optimize for various parameters such as water uptake, therapeutic agent release and/or polymer degradation kinetics can be optimized.

It is contemplated that a disclosed nanoparticle that includes PEG, e.g., includes PLA-PEG, the PEG portion may be terminated and include an end group, for example, when PEG is not conjugated to a ligand. For example, PEG may terminate in a hydroxyl, a methoxy or other alkoxyl group, a methyl or other alkyl group, an aryl group, a carboxylic acid, an amine, an amide, an acetyl group, a guanidino group, or an imidazole. Other contemplated end groups include azide, alkyne, maleimide, aldehyde, hydrazide, hydroxylamine, alkoxyamine, or thiol moieties.

In some embodiments nanoparticles include a poly(lactic) acid-poly(ethylene)glycol copolymer having a poly(lactic) acid number average molecular weight fraction of about 0.6 to about 0.95, in some embodiments between about 0.7 to about 0.9, in some embodiments between about 0.6 to about 0.8, in some embodiments between about 0.7 to about 0.8, in some embodiments between about 0.75 to about 0.85, in some embodiments between about 0.8 to about 0.9, and in some embodiments between about 0.85 to about 0.95. It should be understood that the poly(lactic) acid number average molecular weight fraction may be calculated by dividing the number average molecular weight of the poly(lactic) acid component of the copolymer by the sum of the number average molecular weight of the poly(lactic) acid component and the number average molecular weight of the poly(ethylene)glycol component.

Those of ordinary skill in the art will know of methods and techniques for PEGylating a polymer, for example, by using EDC (1-ethyl-3-(3-dimethylaminopropyl) carbodiimide hydrochloride) and NHS (N-hydroxysuccinimide) to react a polymer to a PEG group terminating in an amine, by ring opening polymerization techniques (ROMP), or the like

A disclosed particle can for example comprise a diblock copolymer of PEG and PL(G)A, wherein for example, the PEG portion may have a number average molecular weight of about 1,000-20,000, e.g., about 2,000-20,000, e.g., about 5 kDa, and the PL(G)A portion may have a number average molecular weight of about 5,000 to about 20,000, or about 5,000-100,000, e.g., about 20,000-70,000, e.g., about 15,000-50,000, e.g., about 15 kDa.

For example, disclosed here is an exemplary therapeutic nanoparticle that includes about 10 to about 99 weight percent poly(lactic) acid-poly(ethylene)glycol copolymer or poly(lactic)-co-poly (glycolic) acid-poly(ethylene)glycol copolymer, or about 20 to about 80 weight percent, about 40 to about 80 weight percent, or about 30 to about 50 weight percent, or about 70 to about 90 weight percent poly(lactic) acid-poly(ethylene)glycol copolymer or poly(lactic)-co-poly (glycolic) acid-poly(ethylene)glycol copolymer. Exemplary poly(lactic) acid-poly(ethylene)glycol copolymers can include a number average molecular weight of about 15 to about 20 kDa, or about 10 to about 25 kDa of poly(lactic) acid and a number average molecular weight of about 4 to about 6, or about 2 kDa to about 10 kDa of poly(ethylene)glycol.

Disclosed nanoparticles may optionally include about 1 to about 50 weight percent poly(lactic) acid or poly(lactic) acid-co-poly (glycolic) acid (which does not include PEG), or may optionally include about 1 to about 50 weight percent, or about 10 to about 50 weight percent or about 30 to about 50 weight percent poly(lactic) acid or poly(lactic) acid-co-poly (glycolic) acid. For example, poly(lactic) or poly(lactic)-co-poly(glycolic) acid may have a number average molecule weight of about 5 to about 15 kDa, or about 5 to about 12 kDa. Exemplary PLA may have a number average molecular weight of about 5 to about 10 kDa. Exemplary PLGA may have a number average molecular weight of about 8 to about 12 kDa.

Disclosed nanoparticles that may include an optional targeting moiety, i.e., a moiety able to bind to or otherwise associate with a biological entity, for example, a membrane component, a cell surface receptor, prostate specific membrane antigen, or the like. A targeting moiety present on the surface of the particle may allow the particle to become localized at a particular targeting site, for instance, a tumor, a disease site, a tissue, an organ, a type of cell, etc. As such, the nanoparticle may then be “target specific.” The drug or other payload may then, in some cases, be released from the particle and allowed to interact locally with the particular targeting site.

In one embodiment, a disclosed nanoparticle includes a targeting moiety that is a low-molecular weight ligand, e.g., a low-molecular weight PSMA ligand. The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones, or the like. The term “binding partner” refers to a molecule that can undergo binding with a particular molecule. “Specific binding” refers to molecules, such as polynucleotides, that are able to bind to or recognize a binding partner (or a limited number of binding partners) to a substantially higher degree than to other, similar biological entities. In one set of embodiments, the targeting moiety has an affinity (as measured via a disassociation constant) of less than about 1 micromolar, at least about 10 micromolar, or at least about 100 micromolar.

For example, a targeting portion may cause the particles to become localized to a tumor (e.g. a solid tumor) a disease site, a tissue, an organ, a type of cell, etc. within the body of a subject, depending on the targeting moiety used. For example, a low-molecular weight PSMA ligand may become localized to a solid tumor, e.g. pancreas tumors or cancer cells. The subject may be a human or non-human animal. Examples of subjects include, but are not limited to, a mammal such as a dog, a cat, a horse, a donkey, a rabbit, a cow, a pig, a sheep, a goat, a rat, a mouse, a guinea pig, a hamster, a primate, a human or the like.

For example a target moiety may be PSMA peptidase inhibitor moieties, for example, a ligand represented by:

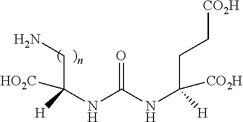

and enantiomers, stereoisomers, rotamers, tautomers, diastereomers, or racemates thereof, wherein n is 1, 2, 3, 4, 5 or 6. For this ligand, the NH₂ group serves as the point of covalent attachment to the nanoparticle (e.g., —N(H)—PEG).

In another embodiment, a disclosed nanoparticle may include a PEG-PLA copolymer bound to a low-molecular weight PSMA ligand represented by:

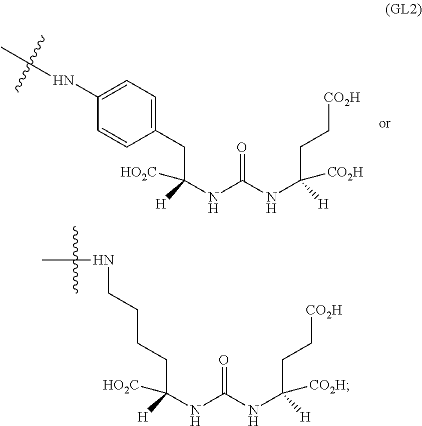 [SMILES: *Nc1ccc(C[C@H](NC(=O)N[C@@H](CCC(=O)O)C(=O)O)C(=O)O)cc1 ; *NCCCC[C@H](NC(=O)N[C@@H](CCC(=O)O)C(=O)O)C(=O)O]

and enantiomers, stereoisomers, rotamers, tautomers, diastereomers, or racemates thereof.

Targeting moieties disclosed herein are typically conjugated to a disclosed polymer or copolymer (e.g. PLA-PEG), and such a polymer conjugate may form part of a disclosed nanoparticle. For example, a disclosed therapeutic nanoparticle may optionally include about 0.2 to about 10 weight percent of a PLA-PEG or PLGA-PEG, wherein the PEG is functionalized with a targeting ligand (e.g. PLA-PEG-Ligand). Contemplated therapeutic nanoparticles may include, for example, about 0.2 to about 10 mole percent PLA-PEG-GL2 or poly (lactic) acid-co poly (glycolic) acid-PEG-GL2. For example, PLA-PEG-GL2 may include a number average molecular weight of about 10 kDa to about 20 kDa and a number average molecular weight of about 4,000 to about 8,000.

Such a targeting ligand may be, in some embodiments, covalently bound to the PEG, for example, bound to the PEG via an alkylene linker, e.g. PLA-PEG-alkylene-GL2. For example, a disclosed nanoparticle may include about 0.2 to about 10 mole percent PLA-PEG-GL2 or poly (lactic) acid-co poly (glycolic) acid-PEG-GL2. It is understood that reference to PLA-PEG-GL2 or PLGA-PEG-GL2 refers to moieties that may include an alkylene linker (e.g. C₁-C₂₀, e.g., (CH₂)₅) linking a PLA-PEG or PLGA-PEG to GL2.

Disclosed nanoparticles may include an exemplary polymeric conjugates such as one of:

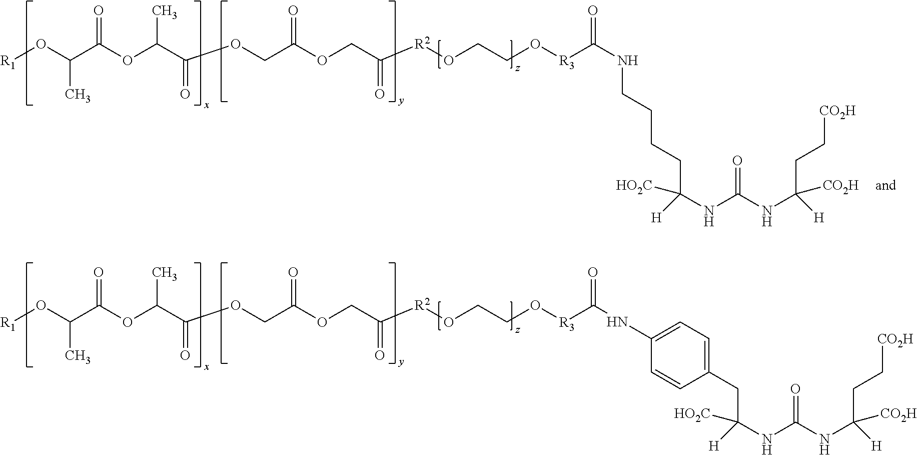

wherein R₁ is selected from the group consisting of H, and a C₁-C₂₀ alkyl group optionally substituted with one, two, three or more halogens;

R₂ is a bond, an ester linkage, or amide linkage;

R₃ is an C₁-C₁₀ alkylene or a bond;

x is 50 to about 1500, or about 60 to about 1000;

y is 0 to about 50, and

z is about 30 to about 200, or about 50 to about 180.

In a different embodiment, x represents 0 to about 1 mole fraction; and y may represent about 0 to about 0.5 mole fraction. In an exemplary embodiment, x+y may be about 20 to about 1720, and/or z may be about 25 to about 455.

For example, a disclosed nanoparticle may include a polymeric targeting moiety represented by Formula VI:

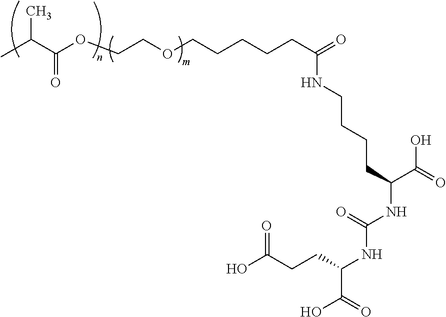

wherein n is about 200 to about 300, e.g., about 222, and m is about 80 to about 130, e.g. about 114. Disclosed nanoparticles, in certain embodiments, may include about 0.1 to about 4% by weight of e.g. a polymeric conjugate of formula VI, or about 0.1 to about 2% or about 0.1 to about 1%, or about 0.2% to about 0.8% by weight of e.g., a polymeric conjugate of formula VI, e.g., about 2.25 weight percent of a disclosed nanoparticle. In another embodiment, a polymeric targeting ligand of formula VI may be about 2.5% by weight of the total polymer included in a disclosed polymer. For example, a disclosed nanoparticle may include about 80-90% by weight polymer component, wherein the polymer component includes about 96-98 weight percent PLA-PEG (e.g. 16 kDa PEG/5 kDa PLA), and about 2-3 weight percent PLA-PEG-Ligand, e.g., PLA-PEG-GL2, (e.g. 16 kDa PEG/5 kDa PLA, e.g. formula VI).

In an exemplary embodiment, a disclosed nanoparticle comprises a nanoparticle having a PLA-PEG-alkylene-GL2 conjugate, where, for example, PLA has a number average molecular weight of about 16,000 Da, PEG has a molecular weight of about 5000 Da, and e.g., the alkylene linker is a C₁-C₂₀ alkylene, e.g. (CH₂)₅.

For example, a disclosed nanoparticle may include a conjugate represented by:

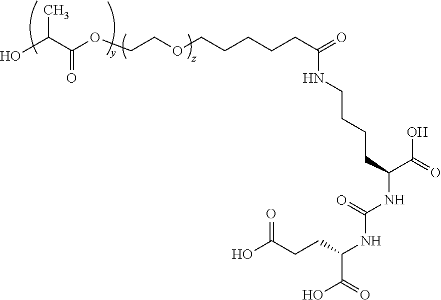

where y is about 222 and z is about 114. A disclosed polymeric conjugate may be formed using any suitable conjugation technique. Disclosed nanoparticles may have a substantially spherical (i.e., the particles generally appear to be spherical), or non-spherical configuration. For instance, the particles, upon swelling or shrinkage, may adopt a non-spherical configuration. In some cases, the particles may include polymeric blends. For instance, a polymer blend may be formed that includes a first polymer comprising a targeting moiety (i.e., a low-molecular weight PSMA ligand) and a biocompatible polymer, and a second polymer comprising a biocompatible polymer but not comprising the targeting moiety. By controlling the ratio of the first and second polymers in the final polymer, the concentration and location of targeting moiety in the final polymer may be readily controlled to any suitable degree.

Disclosed nanoparticles may have a characteristic dimension of less than about 1 micrometer, where the characteristic dimension of a particle is the diameter of a perfect sphere having the same volume as the particle. For example, the particle can have a characteristic dimension of the particle can be less than about 300 nm, less than about 200 nm, less than about 150 nm, less than about 100 nm, less than about 50 nm, less than about 30 nm, less than about 10 nm, less than about 3 nm, or less than about 1 nm in some cases. In particular embodiments, the nanoparticle of the present invention has a diameter of about 80 nm-200 nm, about 60 nm to about 150 nm, or about 70 nm to about 200 nm.

In an embodiment, a disclosed nanoparticle can comprise a first diblock polymer comprising a poly(ethylene glycol) and a targeting moiety conjugated to the poly(ethylene glycol), and a second polymer comprising the poly(ethylene glycol) but not the targeting moiety, or comprising both the poly(ethylene glycol) and the targeting moiety, where the poly(ethylene glycol) of the second polymer has a different length (or number of repeat units) than the poly(ethylene glycol) of the first polymer. As another example, a particle may comprise a first polymer comprising a first biocompatible portion and a targeting moiety, and a second polymer comprising a second biocompatible portion different from the first biocompatible portion (e.g., having a different composition, a substantially different number of repeat units, etc.) and the targeting moiety. As yet another example, a first polymer may comprise a biocompatible portion and a first targeting moiety, and a second polymer may comprise a biocompatible portion and a second targeting moiety different from the first targeting moiety.

For example, disclosed herein is a therapeutic polymeric nanoparticle capable of binding to a target, comprising a first non-functionalized polymer; an optional second non-functionalized polymer; a functionalized polymer comprising a targeting moiety; and a therapeutic agent; wherein said nanoparticle comprises about 15 to about 300 molecules of functionalized polymer, or about 20 to about 200 molecules, or about 3 to about 100 molecules of functionalized polymer.

Disclosed nanoparticles may be stable (e.g. retain substantially all active agent) for example in a solution that may contain a saccharide, for at least about 3 days, about 4 days or at least about 5 days at room temperature, or at 25° C.

In some embodiments, disclosed nanoparticles may also include a fatty alcohol, which may increase the rate of drug release. For example, disclosed nanoparticles may include a C₈-C₃₀ alcohol such as cetyl alcohol, octanol, stearyl alcohol, arachidyl alcohol, docosonal, or octasonal.

In a particular embodiment, a disclosed nanoparticle composition comprises nanoparticles having a hydrodynamic diameter of about 60 to about 130 nm. Such nanoparticles may include, for example, a chemotherapeutic agent (e.g. about 10 weight percent docetaxel) and about 90 weight percent of a polymer composition. The polymer composition may comprise about 97.5 weight percent diblock poly(lactic)acid-co-poly(ethylene)glycol (with e.g., the poly(lactic acid) block having a number average molecular weight of about 15 to 20 kDa and the poly(ethylene)glycol block has a number average molecular weight of about 4 to about 6 kDa) and about 2.5 weight percent PLA-PEG-GL2 (with e.g., the poly(lactic acid) block having a number average molecular weight of about 15 to 20 kDa and the poly(ethylene)glycol block has a number average molecular weight of about 4 to about 6 kDa, e.g. 15 kDa/5 kDa PLA/PEG-GL2.

Nanoparticles may have controlled release properties, e.g., may be capable of delivering an amount of active agent to a patient, e.g., to specific site in a patient, over an extended period of time, e.g. over 1 day, 1 week, or more. In some embodiments, disclosed nanoparticles substantially immediately releases (e.g. over about 1 minute to about 30 minutes) less than about 2%, less than about 5%, or less than about 10% of an active agent (e.g. a taxane) agent, for example when places in a phosphate buffer solution at room temperature and/or at 37° C.

For example, disclosed nanoparticles that include a therapeutic agent, may, in some embodiments, may release the therapeutic agent when placed in an aqueous solution at e.g., 25 C with a rate substantially corresponding to a) from about 0.01 to about 20% of the total therapeutic agent is released after about 1 hour; b) from about 10 to about 60% of the therapeutic agent is released after about 8 hours; c) from about 30 to about 80% of the total therapeutic agent is released after about 12 hours; and d) not less than about 75% of the total is released after about 24 hours.

In some embodiments, after administration to a subject or patient of a disclosed nanoparticle or a composition that includes a disclosed nanoparticle, the peak plasma concentration (Cₘₐₓ) of the therapeutic agent in the patient s substantially higher as compared to a Cₘₐₓ of the therapeutic agent if administered alone (e.g., not as part of a nanoparticle).

In another embodiment, a disclosed nanoparticle including a therapeutic agent, when administered to a subject, may have a tₘₐₓ of therapeutic agent substantially longer as compared to a tₘₐₓ of the therapeutic agent administered alone.

### Therapeutic Agents

Disclosed nanoparticles may include a therapeutic agent such as an antineoplastic agent, e.g. such as a mTor inhibitor (e.g., sirolimus, temsirolimus, or everolimus), a vinca alkaloid such as vincristine, a diterpene derivative or a taxane such as paclitaxel (or its derivatives such as DHA-paclitaxel or PG-paxlitaxel) or docetaxel.

In one set of embodiments, a disclosed nanoparticle may include a drug or a combination of more than one drug. Such particles may be useful, for example, in embodiments where a targeting moiety may be used to direct a particle containing a drug to a particular localized location within a subject, e.g., to allow localized delivery of the drug to occur. Exemplary therapeutic agents include chemotherapeutic agents such as doxorubicin (adriamycin), gemcitabine (gemzar), daunorubicin, procarbazine, mitomycin, cytarabine, etoposide, methotrexate, vinorelbine, 5-fluorouracil (5-FU), vinca alkaloids such as vinblastine or vincristine; bleomycin, paclitaxel (taxol), docetaxel (taxotere), aldesleukin, asparaginase, carboplatin, cladribine, camptothecin, 10-hydroxy-7-ethylcamptothecin (SN38), dacarbazine, S-I capecitabine, 5′deoxyflurouridine, eniluracil, deoxycytidine, 5-azacytosine, 5-azadeoxycytosine, allopurinol, 2-chloroadenosine, trimetrexate, aminopterin, methylene-10-deazaaminopterin (MDAM), oxaplatin, picoplatin, ormaplatin, epirubicin, etoposide phosphate, 9-aminocamptothecin, 10,11-methylenedioxycamptothecin, karenitecin, 9-nitrocamptothecin, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, and combinations thereof. Non-limiting examples of potentially suitable drugs include anti-cancer agents, including, for example, docetaxel, mitoxantrone, and mitoxantrone hydrochloride.

Nanoparticles disclosed herein may be combined with pharmaceutical acceptable carriers to form a pharmaceutical composition, according to another aspect of the invention. As would be appreciated by one of skill in this art, the carriers may be chosen based on the route of administration as described below, the location of the target issue, the drug being delivered, the time course of delivery of the drug, etc.

The pharmaceutical compositions of this invention can be administered to a patient by any means known in the art including oral and parenteral routes. The term “patient,” as used herein, refers to humans as well as non-humans, including, for example, mammals, birds, reptiles, amphibians, and fish. For instance, the non-humans may be mammals (e.g., a rodent, a mouse, a rat, a rabbit, a monkey, a dog, a cat, a primate, or a pig). In certain embodiments parenteral routes are desirable since they avoid contact with the digestive enzymes that are found in the alimentary canal. According to such embodiments, inventive compositions may be administered by injection (e.g., intravenous, subcutaneous or intramuscular, intraperitoneal injection), rectally, vaginally, topically (as by powders, creams, ointments, or drops), or by inhalation (as by sprays).

In a particular embodiment, the nanoparticles of the present invention are administered to a subject in need thereof systemically, e.g., by IV infusion or injection.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. In one embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In some embodiments, a composition suitable for freezing is contemplated, including nanoparticles disclosed herein and a solution suitable for freezing, e.g. a sucrose and/or cyclodextrin solution is added to the nanoparticle suspension. The sucrose may e.g., as a cryoprotectant to prevent the particles from aggregating upon freezing. For example, provided herein is a nanoparticle formulation comprising a plurality of disclosed nanoparticles, sucrose and and water.

### Methods of Treatment

In some embodiments, targeted particles in accordance with the present invention may be used to treat, alleviate, ameliorate, relieve, delay onset of, inhibit progression of, reduce severity of, and/or reduce incidence of one or more symptoms or features of a disease, disorder, and/or condition.

In one embodiment, the disclosure provides a method of treating certain cancers such as cancers of lymph or biliary ducts or biliary tract, (e.g. cholangiocarcinoma, pancreatic cancer, gallbladder cancer, and/or cancer of the ampulla of Vater), comprising administering to a patient in need thereof a composition comprising a disclosed therapeutic nanoparticle (e.g. a nanoparticle comprising a therapeutic agent (e.g. docetaxel) and a biocompatible polymer).

In another embodiment, the disclosure provides a method of treating certain cancers such as oropharyngeal cancers, cancers of the head and neck, or cancers of the throat, e.g. tonsillar cancer, comprising administering to a patient in need thereof: a composition comprising a disclosed therapeutic nanoparticle (e.g. a nanoparticle comprising a therapeutic agent (e.g. docetaxel) and a biocompatible polymer).

Also provided herein are methods of treating gastrointestinal cancers such as anal cancer, colorectal cancer, pancreatic cancer, gastrointestinal stromal tumors, esophageal cancer, liver cancer, gallbladder cancer and/or cancer of the bowel. Disclosed here, in some embodiments, is a method of treating cervical cancer, prostate cancer, bladder cancer and/or lung cancer (e.g. small cell or non small cell lung cancer (e.g. adenocarcinoma, squamous cell carcinoma) and/or breast cancer, using, for example, disclosed dosages of therapeutic nanoparticle compositions.

Also provided herein are methods of administering to a patient a nanoparticle disclosed herein including an active agent, wherein, upon administration to a patient, such nanoparticles substantially reduces the volume of distribution and/or substantially reduces free Cmax, as compared to administration of the agent alone (i.e. not as a disclosed nanoparticle).

Disclosed methods may include administration of a disclosed nanoparticle composition, wherein the composition is administered over a period of three weeks, a month, or two months or more. For example, disclosed herein are methods of treating cancers that include administering a disclosed nanoparticle composition over a period of at least two weeks, three weeks, one month or administered over a period of about 2 weeks to about 6 months or more, wherein the interval between each administration is no more than about once a day, once a week, once every two weeks, once every three weeks, or once every month, and wherein the dose of the active agent (e.g. docetaxel) at each administration is about 30 mg/m² to about 75 mg/m², or about 50 mg/m² to about 75 mg/m², or about 60 mg/m² to about 70 mg/m² or about 55 mg/m² or about 60 mg/m².

Provided herein is a method of treating a cancer (e.g. refractory cancer) in a patient in need thereof, comprising administering to the patient a therapeutically effective amount of a disclosed nanoparticle composition. Such a refractory cancer may be e.g., gastrointestinal cancer, oropharyngeal cancer, cervical cancer, lung cancer, or prostate cancer, for example, a refractory cancer may be tonsillar cancer, anal cancer, pancreatic cancer, bile duct cancer, colon cancer, cervical cancer, or gallbladder cancer. A refractory cancer may be a cancer that has been treated previously in a patient with one or more chemotherapeutics and/or radiation, but that is not responsive to those first line therapies.

Contemplated herein are method of treating cancers, e.g. refractory cancers, in a patient comprising administering a) an effective amount of a disclosed nanoparticle composition comprising a therapeutic agent (e.g. docetaxel) and optionally b) an effective amount of at least one other chemotherapeutic agent. In some embodiments, the other chemotherapeutic agent is cisplatin, capecitabine, oxaliplatin, gemcitabine, 5FU, mitomycin, gemcitabine or a combination of other chemotherapeutic agents. In such combination therapies, the composition comprising nanoparticles and the other chemotherapeutic agent can be administered simultaneously, either in the same composition or in separate compositions, administered sequentially, i.e., the nanoparticle composition can be administered either prior to or after the administration of the other chemotherapeutic agent. In some embodiments, the administration of the nanoparticle composition and the chemotherapeutic agent can be concurrent, i.e., the administration period of the nanoparticle composition and that of the chemotherapeutic agent overlap with each other. In some embodiments, the administration of the nanoparticle composition and the chemotherapeutic agent are non-concurrent. For example, in some embodiments, the administration of the nanoparticle composition is terminated before the chemotherapeutic agent is administered. In some embodiments, the administration of the other chemotherapeutic agent is terminated before the nanoparticle composition is administered. In a method of treating refractory cancer, the patient may not have been responsive to the other agents.

Methods of treating cancer are also contemplated that a) a first therapy comprising administering to a patient a disclosed nanoparticle composition, and b) a second therapy comprising radiation therapy, surgery, or combinations thereof.

Provided herein, in some embodiments, are methods of treating cancers that are refractory to other chemotherapeutic agents, for example, 5FU alone or in combination with chemotherapys or radiation therapy. For example, provided herein is a method of treating a refractory cervical cancer in a patient need thereof, wherein the patient was not responsive to cisplatin or radiation therapy, comprising administering to the patient a therapeutically effective amount of a composition comprising therapeutic nanoparticles, (e.g. administering about 50 to about 75 mg/m² of docetaxel), wherein said nanoparticles comprise about 10 weight percent docetaxel and a poly(lactic) acid-poly(ethylene)glycol diblock copolymer.

### EXAMPLES

The invention now being generally described, it will be more readily understood by reference to the following examples which are included merely for purposes of illustration of certain aspects and embodiments of the present invention, and are not intended to limit the invention in any way.

### Example 1: Nanoparticle Preparation—Emulsion Process

An organic phase is formed composed of a mixture of docetaxel (DTXL) and polymer (co-polymer, and/or co-polymer with ligand). The organic phase is mixed with an aqueous phase at approximately a 1:5 ratio (oil phase:aqueous phase) where the aqueous phase is composed of a surfactant and some dissolved solvent. In order to achieve high drug loading, about 30% solids in the organic phase is used.

The primary, coarse emulsion is formed by the combination of the two phases under simple mixing or through the use of a rotor stator homogenizer. The rotor/stator yielded a homogeneous milky solution, while the stir bar produced a visibly larger coarse emulsion. It was observed that the stir bar method resulted in significant oil phase droplets adhering to the side of the feed vessel, suggesting that while the coarse emulsion size is not a process parameter critical to quality, it should be made suitably fine in order to prevent yield loss or phase separation. Therefore the rotor stator is used as the standard method of coarse emulsion formation, although a high speed mixer may be suitable at a larger scale.

The primary emulsion is then formed into a fine emulsion through the use of a high pressure homogenizer. The size of the coarse emulsion does not significantly affect the particle size after successive passes (103) through the homogenizer M-110-EH.

Homogenizer feed pressure was found to have a significant impact on resultant particle size. On both the pneumatic and electric M-110EH homogenizers, it was found that reducing the feed pressure also reduced the particle size. Therefore the standard operating pressure used for the M-110EH is 4000-5000 psi per interaction chamber, which is the minimum processing pressure on the unit. The M-110EH also has the option of one or two interaction chambers. It comes standard with a restrictive Y-chamber, in series with a less restrictive 200 μm Z-chamber. It was found that the particle size was actually reduced when the Y-chamber was removed and replaced with a blank chamber. Furthermore, removing the Y-chamber significantly increases the flow rate of emulsion during processing.

After 2-3 passes the particle size was not significantly reduced, and successive passes can even cause a particle size increase. Table A summarizes the emulsification process parameters.

TABLE A

| Parameter | Value | Observation |
|---|---|---|
| Coarse | Rotor stator | Coarse emulsion size does not affect |
| emulsion | homogenizer | final particle size, but large |
| formation | | coarse emulsion can cause increased |
| | | oil phase retention in feed vessel |
| Homogenizer | 4000-5000 psi | Lower pressure reduces particle size |
| feed | per chamber | |
| pressure | | |
| Interaction | 2 × 200 μm | 200 μm Z-chamber yields the smallest |
| chamber(s) | Z-chamber | particle size, and allows for |
| | | highest homogenizer throughput |
| Number of | 2-3 passes | Studies have shown that the particle |
| homogenizer | | size is not significantly reduced |
| passes | | after 2 discreet passes, and size can |
| | | even increase with successive passes |
| Water phase | 0.1% | [Sodium cholate] can effectively |
| [sodium | | alter particle size; value is optimized |
| cholate] | | for given process and formulation |
| W:O ratio | 5:1 | Lowest ratio without significant particle |
| | | size increase is ~5:1 |
| [Solids] | 30% | Increased process efficiency, increased |
| in oil phase | | drug encapsulation, workable viscosity |

The fine emulsion is then quenched by addition to deionized water at a given temperature under mixing. In the quench unit operation, the emulsion is added to a cold aqueous quench under agitation. This serves to extract a significant portion of the oil phase solvents, effectively hardening the nanoparticles for downstream filtration. Chilling the quench significantly improved drug encapsulation. The quench:emulsion ratio is approximately 5:1.

A solution of 35% (wt %) of Tween 80 is added to the quench to achieve approximately 2% Tween 80 overall After the emulsion is quenched a solution of Tween-80 is added which acts as a drug solubilizer, allowing for effective removal of unencapsulated drug during filtration. Table B indicates each of the quench process parameters.

TABLE B
Summary quench process parameters.

| Parameter | Value | Observation |
|---|---|---|
| Initial quench | <5° C. | Low temperature yields higher |
| temperature | | drug encapsulation |
| [Tween-80] | 35% | Highest concentration that |
| solution | | can be prepared and readily |
| | | disperses in quench |
| Tween-80:drug | 25:1 | Minimum amount of Tween-80 |
| ratio | | required to effectively remove |
| | | unencapsulated drug |
| Q:E ratio | 5:1 | Minimum Q:E ratio while retain- |
| | | ing high drug encapsulation |
| Quench hold/ | ≦5° C. (with | Temperature which prevents |
| processing temp | current 5:1 Q:E | significant drug leaching |
| | ratio, 25:1 Tween- | during quench hold time and |
| | 80:drug ratio) | initial concentration step |

The temperature must remain cold enough with a dilute enough suspension (low enough concentration of solvents) to remain below the T_{g} of the particles. If the Q:E ratio is not high enough, then the higher concentration of solvent plasticizes the particles and allows for drug leakage. Conversely, colder temperatures allow for high drug encapsulation at low Q:E ratios (to ˜3:1), making it possible to run the process more efficiently.

The nanoparticles are then isolated through a tangential flow filtration process to concentrate the nanoparticle suspension and buffer exchange the solvents, free drug, and drug solubilizer from the quench solution into water. A regenerated cellulose membrane is used with a molecular weight cutoffs (MWCO) of 300.

A constant volume diafiltration (DF) is performed to remove the quench solvents, free drug and Tween-80. To perform a constant-volume DF, buffer is added to the retentate vessel at the same rate the filtrate is removed. The process parameters for the TFF operations are summarized in Table C. Crossflow rate refers to the rate of the solution flow through the feed channels and across the membrane. This flow provides the force to sweep away molecules that can foul the membrane and restrict filtrate flow. The transmembrane pressure is the force that drives the permeable molecules through the membrane.

TABLE C
TFF Parameters

| | Optimized | | |
|---|---|---|---|
| Parameter | Value | | Effect |
| Membrane | Regenerated | | No difference in performance |
| Material | cellulose - | | between RC and PES, but solvent |
| | Coarse Screen | | compatibility is superior for RC. |
| | Membrane | | |
| Molecular | 300 | kDa | No difference in NP characteristics |
| Weight | | | (i.e. residual tween)Increase in flux |
| Cut off | | | rates is seen with 500 kDa membrane |
| | | | but 500 kDa is not available in RC |
| Crossflow | 11 | L/min/m² | Higher crossflow rate led to higher |
| Rate | | | flux |
| Trans- | 20 | psid | Open channel membranes have |
| membrane | | | maximum flux rates between |
| Pressure | | | 10 and 30 psid. Coarse channel |
| | | | membranes have maximum flux |
| | | | rates with min TMP (~20 psid). |
| Concentration | 30 | mg/ml | Diafiltration is most efficient |
| of | | | at [NP] ~50 mg/ml with open |
| Nanoparticle | | | channel TFF membranes based on |
| Suspension for | | | flux rates and throughput. |
| Diafiltration | | | With coarse channel membranes |
| | | | the flux rate is optimized at ~30 |
| | | | mg/ml in the starting buffer. |
| Number of | ≧15 (based on | | About 15 diavolumes are needed to |
| Diavolumes | flux increase) | | effectively remove tween-80. End |
| | | | point of diafiltration is determined |
| | | | by in-process control (flux |
| | | | increase plateau). |
| Membrane | ~1 | m²/kg | Membranes sized based on |
| Area | | | anticipated flux rates and |
| | | | volumes required. |

The filtered nanoparticle slurry is then thermal cycled to an elevated temperature during workup. A small portion (typically 5-10%) of the encapsulated drug is released from the nanoparticles very quickly after its first exposure to 25° C. Because of this phenomenon, batches that are held cold during the entire workup are susceptible to free drug or drug crystals forming during delivery or any portion of unfrozen storage. By exposing the nanoparticle slurry to elevated temperature during workup, this ‘loosely encapsulated’ drug can be removed and improve the product stability at the expense of a small drop in drug loading. Table D summarizes two examples of 25° C. processing. Other experiments have shown that the product is stable enough after ˜2-4 diavolumes to expose it to 25° C. without losing the majority of the encapsulated drug. 5 diavolumes is used as the amount for cold processing prior to the 25° C. treatment.

TABLE D

| | | Lots A | Lots B |
|---|---|---|---|
| Drug load | Cold workup | 11.3% | 9.7% |
| | 25° C. workup¹ | 8.7-9.1% | 9.0-9.9% |
| Stability² | Cold workup | <1 day | <1 day |
| | 25° C. workup¹ | 5-7 days | 2-7 days |
| In vitro burst³ | Cold workup | ~10% | Not |
| | 25° C. workup¹ | ~2% | performed |

¹25° C. workup sublots were exposed to 25° C. after at least 5 diavolumes for various periods of time. Ranges are reported because there were multiple sublots with 25° C. exposure.
²Stability data represents the time that final product could be held at 25° C. at 10-50 mg/ml nanoparticle concentrations prior to crystals forming in the slurry (visible by microscopy)
³In vitro burst represents the drug released at the first time point (essentially immediately)

After the filtration process the nanoparticle suspension is passed through a sterilizing grade filter (0.2 μm absolute). Pre-filters are used to protect the sterilizing grade filter in order to use a reasonable filtration area/time for the process. Values are as summarized in Table E.

TABLE E

| Parameter | O Value | | Effect |
|---|---|---|---|
| Nanoparticle | 50 | mg/ml | Yield losses are higher at higher |
| Suspension | | | [NP], but the ability to |
| Concentration | | | filter at 50 mg/ml obviates the |
| | | | need to aseptically concentrate |
| | | | after filtration |
| Filtration | ~1.3 | L/min/m² | Filterability decreases as flow |
| flow rate | | | rate increases |

The filtration train is Ertel Alsop Micromedia XL depth filter M953P membrane (0.2 μm Nominal); Pall SUPRAcap with Seitz EKSP depth filter media (0.1-0.3 μm Nominal); Pall Life Sciences Supor EKV 0.65/0.2 micron sterilizing grade PES filter.

0.2 m² of filtration surface area per kg of nanoparticles for depth filters and 1.3 m2 of filtration surface area per kg of nanoparticles for the sterilizing grade filters can be used.

### Example 2—Treatment of Human Cholangiocarcinoma

Tumor size following intravenous administration of docetaxel using nanoparticles prepared as in Example 1 (10 wt % docetaxel, 90 wt polymer (˜2.5 wt % PLA-PEG-GL2; and ˜97.5% PLA-PEG, Mn PLA=16 Da; Mn PEG=5 Da; BIND-14) was assessed in a 51 year old human cholangiocarcinoma patient. A dose of 15 mg/m2 docetaxel was administered as BIND-14, with two cycles of treatment. Doses can be administered using a dose escalation protocol.

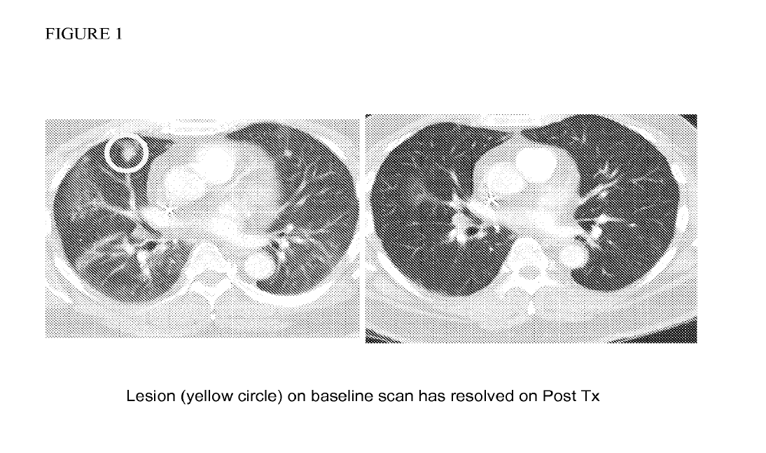

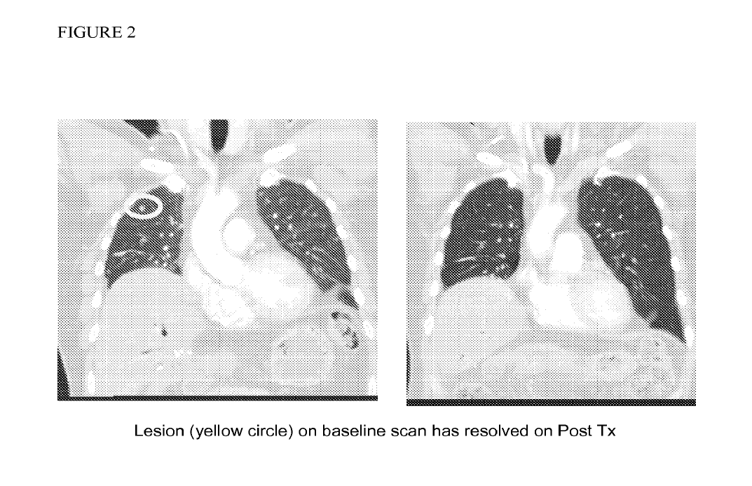

FIGS. 1 and 2 show baseline scans (left panel) and post treatment scans (right panel) taken approximately 6 weeks after treatment with BIND-14. The patient had prior therapy with capecitabin, cisplatin+gemcitabine, oxaliplatin+capecitabine before baseline. The scans indicate that the lesion (circled) was resolved one month post treatment. This result is in contrast to previous studies finding that docetaxel was ineffective for the treatment of cholangiocarcinoma (Pazdur, Am. J. Clin. Oncol. 1999 Volume 22(1), pp 78-81)

### Example 3—Treatment of Human Tonsillar Cancer

Tumor size following intravenous administration of docetaxel using nanoparticles prepared as in Example 1 (10 wt % docetaxel, 90 wt polymer (˜2.5 wt % PLA-PEG-GL2; and ˜97.5% PLA-PEG, Mn PLA=16 Da; Mn PEG=5 Da; BIND-14) was assessed in a 63 year old human tonsillar cancer patient. A dose of 30 mg/m² docetaxel was administered as BIND-14, with two cycles of treatment.

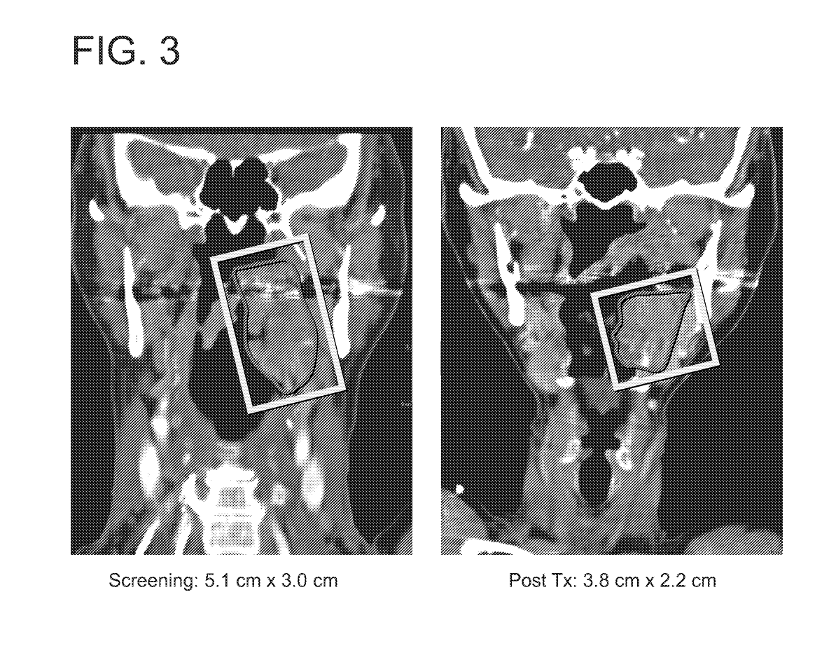

FIG. 3 shows a baseline scan (left panel) and a post treatment scan (right panel) taken approximately 7 weeks after treatment. The patient had prior therapy with four different investigational agents and paclitaxel+carboplatin before baseline. The baseline scan showed a tumor size of 5.1 cm by 3.0 cm, and the post treatment scan showed a tumor size of 3.8 cm by 2.2 cm, indicating that the tumor size decreased following treatment (the tumor is indicated by the rectangles).

### Example 4—Pharmacokinetics of Docetaxel BIND-014 in Human Patients

The pharmacokinetics (PK) of nanoparticles having docetaxel as prepared in Example 1 were determined in human patients. (12 total patients (11 evaluable); male/female 8/4; median age 70 years (29-82); median courses of therapy 1.5 (1-3); previous therapy: chemotherapy (10), prior taxane therapy (4); radiotherapy (4); Tumor type: NSCLC (2 patients); ovarian (1) gastric (1), head and neck (1), other (small-cell lung cancer, cholangiocarcinoma, eccurin, tonsil, adrenocortical, anal cancer). The patients were given a single intravenous dose of passively targeted nanoparticles encapsulating drug (10 wt % drug, 90 wt polymer (PLA-PEG, Mn PLA=16 Da; Mn PEG=5 Da, PTNP) at time=0.

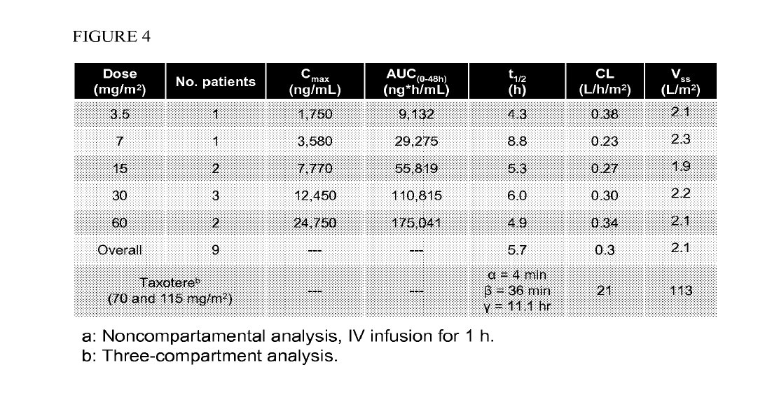

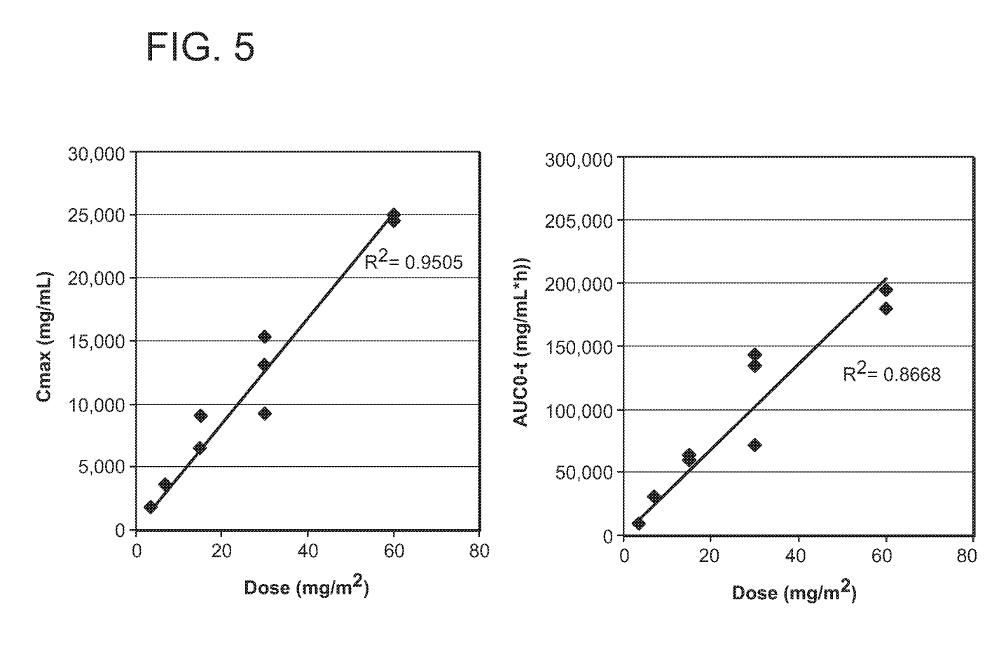

FIG. 4 depicts the PK profiles of docetaxel nanoparticles. FIG. 5 demonstrates the PK linearity. The left panel shows a plot of Cmax as a function of dose, and the right panel shows a plot of the area under the curve for the period t=0 to t=48 hours (AUC(0-48h)) as a function of dose.

### Example 5—Pharmacodynamics of Docetaxel Particles in Human Patients with Advanced Cancer

Nanoparticles having docetaxel (BIND-014) as prepared in Example 1 were determined in human patients. BIND-014 was administered once every three weeks by 1-hour intravenous infusion to patients meeting the main eligibility criteria of >18 years old; advanced or metastatic cancer for which no standard or curative therapy exists; measurable or evaluable disease per RECIST version 1.1.; ECOG performance status 0 or 1; life expectancy >12 weeks. Starting dose was 3.5 mg/m². Each patient received a 60 minute infusion of BIND-14 every 3 weeks.

Dose of 3.5, 7.5, 15, 30, 60 and 75 mg/m² were evaluated in 17 patients (7 to female/10 male; median age 62 years). Transient grade 4 neutropenia was observed in 2/7 patients at 60 mg/m² and ⅔ patients at 75 mg/m². No febrile neutropenia was observed. Non-hematological toxicities were mild to moderate in severity and were well-managed. PK based on measurement of total (encapsulated and released) docetaxel is distinct from sb-docetaxel (solvent-based docetaxel), dose proportional at all doses studied, and consistent with retention of nanoparticles in the plasma compartment and controlled release of docetaxel. Mean CL is 0.3 L/h/m², Vss is 3.6 L/m², and t₁₁₂ is 6 h. SD following >2 cycles of therapy was observed in 1 patient with cholangiocarcinoma at 15 mg/m², 1 patient with tonsillar cancer at 30 mg/m², 1 patient with colorectal cancer at 60 mg/m², 1 patient with anal cancer at 60 mg/m² (durable response with 9 cy) and 1 patient with pancreatic cancer at 75 mg/m² and reduced to 60 mg/m² at cycle 2. A confirmed partial response by RECIST was observed during cycle 1 in a patient with cervical cancer dosed at 75 mg/m².

The table below shows the anti-tumor activity and prior agents administered:

| | Dose | Anti-tumor | |
|---|---|---|---|
| Tumor Type | (mg/m²) | Activity | Prior agents |
| Bile Duct | 15 | Stable Disease | Capecitabine, |
| | | | Cisplatin + Gemcitabine; |
| | | | Oxaliplatin + Capecitabine |
| Tonsil | 30 | Stable Disease | Carboplatin + paclitaxel; |
| | | | Investigational Agents |
| Anus | 60 | Stable Disease | 5FU + Cisplatin |
| (Squamous- | | (26 weeks) | 5FU + Mitomycin |
| Cell) | | | |
| Colon | 60 | Stable Disease | 5FU, |
| | | | FOLFOX + bevacizumab, |
| | | | oxaliplatin, leucovorin, |
| | | | erbitux + irinotecan |
| Pancreas | 75 | Stable Disease | Gemcitabine + erlotinib, |
| | | (18 weeks) | irinotecan + 5FU, |
| | | | Investigational Agent |
| Cervix | 75 | Partial Response | Cisplatin, radiation |
| | | (25+ weeks) | therapy |

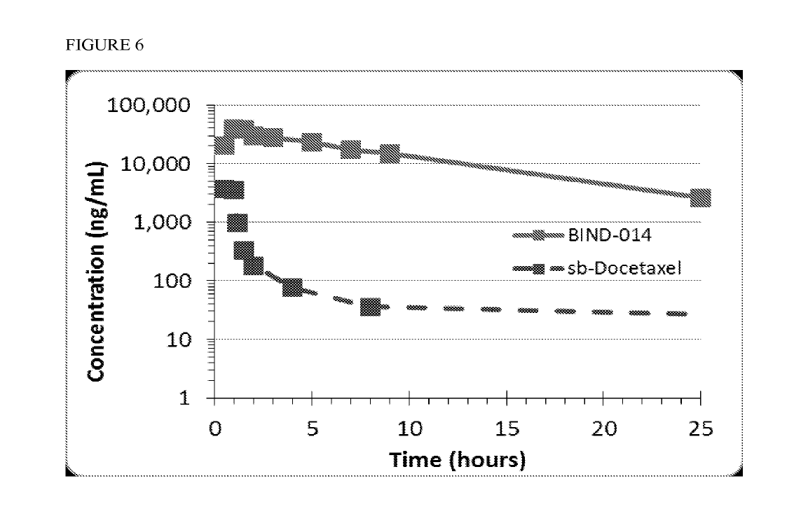

Docetaxel AUCs following BIND-014 administration is more than 100-fold (two orders of magnitude) higher than the same dose of sb-docetaxel. PC profiles were consistent with retention in the plasma compartment and controlled release of docetaxel. FIG. 6 demonstrates the sustained exposure at 75 mg/m² when compared to an equivalent dose of docetaxel alone.

BIND-014 was generally well-tolerated. PK is substantially differentiated from sb-DTXL and preliminary evidence of anti-tumor activity has been observed at low DTLX doses and in tumors in which sb-DTXL has minimal activity.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The entire contents of all patents, published patent applications, websites, and other references cited herein are hereby expressly incorporated herein in their entireties by reference.

What is claimed is:

1. A method of treating cervical cancer in a patient in need thereof, comprising administering to the patient a therapeutically effective amount of a nanoparticle composition, wherein nanoparticle composition comprises nanoparticles having a hydrodynamic diameter of about 60 to about 150 nm comprising:
docetaxel and
about 10 to about 97 weight percent of a diblock poly(lactic) acid-poly(ethylene)glycol copolymer, wherein the poly(lactic acid) block has a number average molecular weight of about 15 to 20 kDa and the poly(ethylene)glycol block has a number average molecular weight of about 4 to about 6 kDa; and
wherein the cervical cancer is a refractory cancer, and wherein the refractory cancer is refractory to other chemotherapy and/or radiation therapy alone.

2. The method of claim 1, wherein the therapeutically effective amount of the nanoparticle composition is about 50 to about 75 mg/m² of docetaxel.

3. The method of claim 2, wherein the therapeutically effective amount of the nanoparticle composition is about 60 to about 75 mg/m² of docetaxel.

4. The method of claim 2, wherein the therapeutically effective amount of the nanoparticle composition is about 60 mg/m² of docetaxel.

5. The method of claim 1, comprising administering the composition about every three weeks to said patient.

6. The method of claim 1, wherein the composition is administered by intravenous infusion over about 1 hour.

7. The method of claim 1, wherein the therapeutically effective amount of the nanoparticle composition is about 75 mg/m² of docetaxel.

8. The method of claim 1, wherein the patient had previously been administered another chemotherapeutic agent and/or radiation.
